(19) United States
(12) Patent Application Publication
(10) Pub. No.: US 2020/0185081 A1
(43) Pub. Date: Jun. 11, 2020

(54) APPLICATION AND METHODS FOR FASTING PROGRAMS AND LIFESTYLE PRACTICE BASED ON AYURVEDIC ELEMENTS, DOSHAS AND THE PRACTICE FOR INTEGRAL LIFE, WELLNESS AND PSYCHIC GROWTH

(71) Applicant: Anal Patel, (US)

(72) Inventor: Anal Patel, Mountain View, CA (US)

(21) Appl. No.: 16/704,614

(22) Filed: Dec. 5, 2019

Related U.S. Application Data
(60) Provisional application No. 62/836,845, filed on Apr. 22, 2019.
(60) Provisional application No. 62/775,490, filed on Dec. 5, 2018.

(51) Int. Cl. G16H 20/90; G16H 10/60; G16H 10/20; G16H 20/30; G16H 20/60; G16H 20/70; G16H 50/20; G01N 33/68; G01N 33/84; G06T 7/90

(52) U.S. Cl. CPC G16H 20/90; G16H 10/60; G16H 10/20; G16H 20/30; G06T 7/90; G16H 20/70; G16H 50/20; G01N 33/6827; G01N 33/84; G16H 20/60

(57) ABSTRACT

A method for designing and monitoring a fasting and lifestyle regimen for a individual can include: a) diagnosing the individual by a series of questions and physical measurements to determine a test profile; b) determining one or more goals to achieve balance by analyzing the test profile of the individual or by accepting direct input of the individual; and c) determining a fasting and lifestyle regimen for the individual by algorithmic mapping of the test profile and one or more goals that can help achieve balance and growth. Test strips for analyzing body fluid are also disclosed, as are methods of use of the test strips. An application for implementing the methods is also disclosed.

### CROSS REFERENCE TO RELATED APPLICATIONS

This application claims priority to U.S. Provisional Patent Application No. 62/775,490, filed Dec. 5, 2018, and U.S. Provisional Patent Application No. 62/836,845, filed Apr. 22, 2019. The contents of each of the foregoing applications are incorporated by reference.

### BACKGROUND

Fasting is a practice of avoiding food consumption, and is utilized worldwide for a variety of purposes. Fasting can be performed, for example, for religious, spiritual, wellness, and health reasons. Many scientific claims have been made linking fasting with a positive impact on human health. Current approaches only define fasting as a method and pattern for food elimination, focusing only on the duration of the fasting based on the personal or communal objectives.

As in other aspects of modern life, technology has been adapted to support new approaches to lifestyle through fasting. Currently marketed technology offers a simple timer-based app to help the end user track time for the start and end of a fasting period, thus focusing only on fasting and relating it to only two criteria: Food, and Time.

Based on the limitations of currently available methodology for diet and fasting, there is a need for methods that integrate food or food avoidance with other components and dimensions impacting human life. Similarly, there is a need for more sophisticated diet and fasting methods beyond simple timing of food consumption, which evolves the perspective to expand from fasting and food to see the life and person as a ‘whole’. There is also a need for disease detection and combination of nutritional care with medicinal care, as well as preventive care before a disease has become evident.

### BRIEF DESCRIPTION OF THE FIGURES

FIG. 1A depicts the overall app structure and app flow for a mobile app for designing and monitoring a fasting and lifestyle regimen.

FIG. 1B depicts the app flow for the diet element of the app.

FIG. 1C depicts the app flow for the meditation element of the app.

FIG. 1D depicts the app flow for the music and story elements of the app.

FIG. 2 depicts a sample summary card fora recipe (lima bean and rice soup).

FIG. 3 depicts a sample summary card for an ingredient (spinach).

FIG. 4 depicts a urine test strip.

FIG. 5 depicts a urine test strip and corresponding color chart.

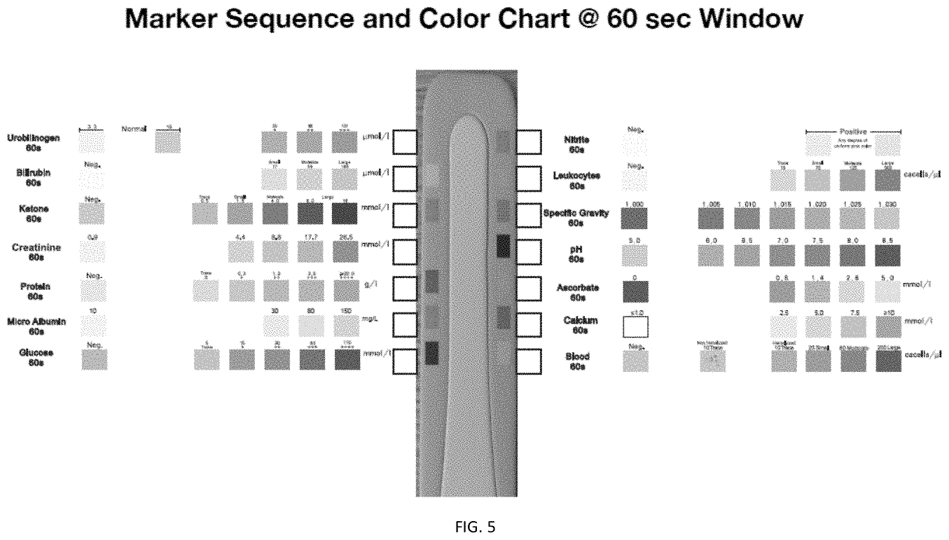

FIG. 6 depicts labels and positions of Chakras.

FIG. 7 depicts levels of subdoshas in each Dosha.

FIG. 8 depicts an example of the degree to which individual ayurvedic elements are contributing to a deranged biomarker in a subject.

FIG. 9 depicts an example of the degree to which individual tissues are in imbalance in a subject.

### DESCRIPTION

The present disclosure includes fasting based on Ayurvedic principles. The word “Ayurveda” is Sanskrit, meaning knowledge of life and longevity. Ayurveda names seven basic tissues (dhatu), which are plasma (rasa), blood (rakta), muscles (mamsa), fat (meda), bone (asthi), marrow (majia), and semen (shukra). Like the medicine of classical antiquity, Ayurveda has historically divided bodily substances into five classical elements (earth, water, fire, air, and ether). There are also 20 points (qualities or characteristics) which are considered to be inherent in all substances. These are organized in ten pairs: Heavy/light, cold/hot, unctuous/dry, dull/sharp, stable/mobile, soft/hard, non-slimy/slimy, smooth/coarse, minute/gross, and viscous/liquid. Ayurveda also names three elemental substances, the doshas (Vata, Pitta, and Kapha), and states that a balance of the doshas results in health, while imbalance results in disease. The Ayurvedic view is that the doshas/elements are balanced when they are equal to each other; that each person should modulate their behavior or environment to increase or decrease the doshas and maintain their natural state using food, meditation, breathing, exercise, sound, sun, and other natural forms of sensory inputs.

Fasting based on the Ayurvedic principles offers an integral solution to an individual's life and serves the need for wellness and growth. The invention intends to serve an individual by developing and offering a guided system for fasting and lifestyle that is not only holistic and integral but also synthesizes the East & the West—the Vedic insight, science and the modern technologies; the esoteric practices and daily lifestyle.

Disclosed herein is the first method and mobile plafform to offer such integral methods of fasting for wellness and lifestyle, with different modules employing the Vedic insight and the Ayurvedic science for body, mind and soul—the “oneness” perspective and consciousness integrating the cosmos, our planet, the human life-mind consciousness, our food and frequencies of sound, time and seasons, in one reality. The invention imparts knowledge about inner processes, realities and different domains of consciousness manifesting in our “outer” habits and personality. Through such integral approach, the platform aims to subtly help transform the individual consciousness.

The invention renews the perspective and practice of fasting, lifestyle, and wellness for contemporary human life, with the following approaches:
Help the individual to engage in fasting as a life-evolving process by allowing them to set up intention not only for a specific day/date but also for personal purpose and need for the program (such as “psycho-emotional detox for anger”, or “nourishing and healing body with nutrition”, etc.).
The service and plafform offer virtual and at-home diagnostics for Ayurvedic profiling to leverage the understanding of the current mental, physical and biological state of the individual, to recommend suitable diet and content modules for unique needs for health and lifestyle.
It offers an opportunity to become aware and prepare the body and mind before initiating a particular fasting program and practice.
Algorithmic mapping of food, music, breathing exercise, meditation, stories, and other formulaic content pursuing the practices and processes espoused in the Ayurvedic paradigm, creates a guided timetable to facilitate engagement with each practice/module at a specific time of the day.
Highly automated and self-learning application to understand user choices, behaviors, intentions, and engagement methods to tailor the service, level, and experience of the individual.
The service and platform help the individual convert aspiration into intention and intention into habit for specific developmental need. This will help the user transcend the self from the current level/domain of consciousness into a higher level/domain of consciousness.
The methods and items disclosed herein can also allow detection of disease states, and allow treatment of existing disease as well as preventive care to avoid future disease.
The designing of a fasting and lifestyle regime can also be combined with medicinal care. The methods and items disclosed herein can be used to design a medicinal treatment regime, e.g., an Ayurvedic herbal medicine regime, to treat the patient. This medicinal treatment regime can be combined with diet, sound therapy, breathing therapy visual video therapy, movement therapy and lifestyle, or the medicinal treatment regimen can be the only prescribed treatment.

A “module” as used herein, is an element of a method.

To “power” the fasting and lifestyle regimen, as used herein, means to design and/or monitor the fasting and lifestyle regimen.

“Program” may be used in place of “regimen” herein.

In one aspect, the disclosure provides a method for designing and monitoring a fasting and lifestyle regimen for an individual (e.g., a person who is interested in integral wellness and is health conscious), the method including: a) diagnosing the individual by a series of questions and physical measurements to determine a test profile; b) determining one or more goals to achieve balance by analyzing the test profile of the individual or by accepting direct input of the individual; c) determining a fasting and lifestyle regimen for the individual by algorithmic mapping of the test profile and one or more goals that can help achieve balance and growth;

wherein the fasting and lifestyle regimen includes specifying i) a duration and method of use of a complete program; and ii) type and amount of food to be eaten when the individual is not in complete fasting. the method further comprising:
a) Determining timing of each activity based on Ayurvedic time scale and solar/lunar cycles;
b) Determining element-based mapping of all activity during fasting and for daily life to help maintain wellness; and
c) Mapping integrated database to power the fasting and lifestyle regimen using Dosha, Elements, Chakras, Seasons, Characteristics, Smell, Taste, Volume and Method of Use;

In some embodiments, the method further comprises d) Determining timing of each activity based on Ayurvedic time scale and solar/lunar cycles; e) Determining element-based mapping of all activity during fasting and for daily life to help maintain wellness; and f) Mapping integrated database to power the fasting and lifestyle regimen using Dosha, Elements, Chakras, Seasons, Characteristics, Smell, Taste, Volume and Method of Use.

In some embodiments, the fasting and lifestyle regimen includes specifying i) a duration and method of use of a complete program; ii) type and amount of Ayurvedically mapped food to be eaten when the individual is not in complete fasting; and iii) one or more modules selected from the group consisting of: music therapy using Ayurvedic principles, pranayama breathing exercises, customized integral yoga meditation, talk, contemplative and story narration, story listening and visual therapy and movement routines, to be performed by the individual.

In some embodiments, the disclosure provides a method for designing and monitoring a fasting and lifestyle regimen for an individual (e.g., a person who is interested in integral wellness and is health conscious), the method including: a) diagnosing the individual by a series of questions and physical measurements to determine a test profile; b) determining one or more goals to achieve balance by analyzing the test profile of the individual or by accepting direct input of the individual; c) determining a fasting and lifestyle regimen for the individual by algorithmic mapping of the test profile and one or more goals that can help achieve balance and growth; d) Determining timing of each activity based on Ayurvedic time scale and solar/lunar cycles; e) Determining element-based mapping of all activity during fasting and for daily life to help maintain wellness; and f) Mapping integrated database to power the fasting and lifestyle regimen using Dosha, Elements, Chakras, Seasons, Characteristics, Smell, Taste, Volume and Method of Use;

wherein the fasting and lifestyle regimen includes specifying i) a duration and method of use of a complete program; ii) type and amount of Ayurvedically mapped food to be eaten when the individual is not in complete fasting; and iii) one or more modules selected from the group consisting of: music therapy using Ayurvedic principles, pranayama breathing exercises, customized integral yoga meditation, talk, contemplative and story narration, story listening and visual therapy and movement routines, to be performed by the individual.

In some embodiments, the algorithmic mapping is performed using Ayurvedic principles, or Ayurvedic principles and sciences. In some embodiments, algorithmic mapping comprises emotional mapping, physical mapping and mental mapping, and wherein physical measurements comprise chemistry and composition of urine, saliva and other body fluids of the patient.

In some embodiments, the fasting and lifestyle regimen comprises (some or all modules) specifying each of music listening, breathing exercises, meditation, story reading, and story listening.

In another aspect, the disclosure provides a system for designing and monitoring a fasting and lifestyle regimen for an individual, the system including executing on a computing device comprising a processor and memory storing instructions that, when executed by the processor, provide an application that performs the following steps: a) collecting a first set of user responses to a series of questions and physical measurements to determine a test profile; b) determining one or more goals to achieve balance, by analyzing the test profile of the individual or by accepting direct input of the individual; c) determining a fasting and lifestyle regimen for the individual, determination including algorithmic mapping of the test profile and one or more goals to achieve balance; f) displaying the actions suggested by the regimen; and g) tracking and displaying the individual's progress in following the regimen; wherein the fasting and lifestyle regimen includes specifying i) a duration of complete fasting; and ii) type and amount of food to be eaten when the individual is not in complete fasting.

In some embodiments, the system includes executing on a computing device comprising a processor and memory storing algorithms that, when executed by the processor, provide an application that performs the following steps: a) collecting a first set of user responses to a series of questions and physical measurements to determine a test profile; b) determining one or more goals to achieve balance, by analyzing the test profile of the individual or by accepting direct input of the individual; c) determining a fasting and lifestyle regimen for the individual, determination including algorithmic mapping of the test profile and one or more goals to achieve balance; d) determining timing of each activity based on Ayurvedic time scale and solar/lunar cycles, and an Ayurvedic element map; e) Determining element-based and chakra-based mapping of all activity during fasting and for daily life to help maintain wellness; f) displaying the actions suggested by the regimen; g) tracking and displaying the individual's progress in following the regimen; and h) accessing a fully mapped integrated database to power the fasting and lifestyle regimen using Elements, Chakras, Doshas, Seasons, Characteristics, Smell, Taste, Volume and Method of Use; wherein the fasting and lifestyle regimen includes specifying i) a duration of complete fasting; ii) type and amount of Ayurvedically mapped food to be eaten when the individual is not in complete fasting; and iii) one or more modules selected from the group consisting of: music listening/therapy, breathing exercises, custom meditation per the program goal, story reading, story/talks listening, and visual video therapy and movement routines to be performed by the individual.

In some embodiments, the system comprises a mobile app. In some embodiments, the system is a mobile app.

In some embodiments, tracking and displaying the individual's progress includes collecting one or more second sets of user responses to a series of questions and physical measurements, to determine a progress profile; and using said progress profile to create a modified fasting and lifestyle regimen.

In some embodiments, the system further comprises algorithmic mapping for deep therapeutic application at a Mind, Body and Soul level to deal with emotions (e.g., anger, fear, etc.), creativity (e.g., intuition, insight, etc.), desires (e.g., sex, jealousy, hate, etc.), physical development (e.g., body vitality, etc.), consciousness (e.g., introspection, contemplation, etc.) and other yogic practices such as SUN Gazing, and Water Fasting.

In some embodiments, collecting a first set of user responses to a series of questions and physical measurements includes collecting a body fluid sample from the individual, and wherein analyzing the body fluid sample produces a body fluid test profile.

In some embodiments, analyzing the body fluid sample includes colorimetric analysis of one or more body fluid test strips to determine levels of one or more biomarkers and thereby produce a body fluid test profile. In some embodiments, colorimetric analysis of the one or more body fluid test strips is performed using a mobile device.

In some embodiments, the system is used on a device comprising a camera, and colorimetric analysis of the one or more body fluid test strips includes
a) taking an image of each test strip using the camera;
b) analysis of the image by the mobile app to determine color hue and/or color density of portions of the test strip, and
c) correlation of the color hue and/or color density with levels of one or more biomarkers to determine levels of each biomarker and thereby produce a body fluid test profile.

In some embodiments, the body fluid is urine or blood. In some embodiments, the body fluid is urine.

In some embodiments, algorithmic mapping of the body fluid test profile is used to detect a disease state present in the individual. In some embodiments, algorithmic mapping of the body fluid test profile is used to determine a treatment to prevent development of a disease in the individual. In some embodiments, algorithmic mapping of the body fluid test profile is used to determine a fasting and lifestyle regimen for the individual. In some embodiments, algorithmic mapping of the body fluid test profile is used to determine a medicinal treatment for the individual. In some embodiments, the medicinal treatment comprises administering Ayurvedic herbal medicine.

In some embodiments, algorithmic mapping of the body fluid test profile comprises determining for each of one or more biomarkers a derangement status based on the extent to which each biomarker level deviates from a pre-determined normal level. In some embodiments, each derangement status is mapped to correlate with an imbalance status of one or more Ayurvedic elements selected from the group consisting of air, earth, fire, water, and ether. In some embodiments, each derangement status is mapped to correlate with an imbalance status of one or more Ayurvedic chakras consisting of solar plexus, sacral, root, heart, and throat/third eye/crown. A derangement status may be mapped to correlate with an imbalance status of only elements, only chakras, or both. In some embodiments, one or more imbalance statuses are used to determine a fasting and lifestyle regimen.

In another aspect, the disclosure provides a urine test strip, the test strip including test portions to determine levels of biomarkers suitable for determining a fasting and lifestyle regimen.

In some embodiments, the biomarkers include one or more selected from the group consisting of urobilinogen, bilirubin, ketone, creatinine, protein, micro albumin, glucose, nitrite, leukocytes, specific gravity, pH, ascorbate, calcium, and blood. In some embodiments, the biomarkers include all of the above-mentioned biomarkers.

In some embodiments, the test strip is suitable for colorimetric analysis by a mobile app, wherein the mobile app is used on a device comprising a camera, and colorimetric analysis of the test strip comprises
a) taking an image of the test strip using the camera;
b) analysis of the image by the mobile app to determine color hue and/or color density of portions of the test strip, and
c) correlation of the color hue and/or color density with levels of one or more biomarkers to determine levels of each biomarker and thereby produce a body fluid test profile.

In another aspect, the disclosure provides a method of analyzing body fluid, the method including:
a) obtaining a sample of body fluid from an individual;
b) exposing a body fluid test strip to the body fluid;
c) taking an image of the test strip using a camera which is part of a mobile device;
d) analysis of the image by a mobile app to determine color hue and/or color density of portions of the test strip, and
e) correlation of the color hue and/or color density with levels of one or more biomarkers to determine levels of each biomarker and thereby produce a body fluid test profile.

In some embodiments, the body fluid is blood or urine. In some embodiments, the body fluid is urine.

In some embodiments, algorithmic mapping of the body fluid test profile is used to detect a disease state present in the individual. In some embodiments, algorithmic mapping of the body fluid test profile is used to determine a treatment to prevent development of a disease in the individual. In some embodiments, algorithmic mapping of the body fluid test profile is used to determine a fasting and lifestyle regimen for the individual. In some embodiments, algorithmic mapping of the body fluid test profile is used to determine a medicinal treatment for the individual. In some embodiments, the medicinal treatment comprises administering Ayurvedic herbal medicine. In some embodiments, algorithmic mapping of the body fluid test profile comprises determining for each of one or more biomarkers a derangement status based on the extent to which each biomarker level deviates from a pre-determined normal level. In some embodiments, each derangement status is mapped to correlate with an imbalance status of one or more Ayurvedic elements selected from the group consisting of air, earth, fire, water, and ether. In some embodiments, each derangement status is mapped to correlate with an imbalance status of one or more Ayurvedic chakras consisting of solar plexus, sacral, root, heart, and throat/third eye/crown. A derangement status may be mapped to correlate with an imbalance status of only elements, only chakras, or both. In some embodiments, one or more imbalance statuses are used to determine a fasting and lifestyle regimen for the individual. In some embodiments, one or more imbalance statuses are used to determine a medicinal treatment regimen for the individual. In some embodiments, one or more imbalance statuses are used to detect or diagnose a disease in the individual. In some embodiments, one or more imbalance statuses are used to determine that an individual is predisposed to a disease or is progressing towards a disease. In some embodiments, one or more imbalance statuses are used to determine a preventive treatment regimen for the individual

Advantageously, the present disclosure provides for a comprehensive technique for designing a multi-faceted fasting and lifestyle regimen which is tailored to an individual's needs and goals. Further provided is a method of building a user-friendly app or platform to collect individual information, and using a proprietary algorithm to prescribe a selection of actions, based on individual specifics, to those which are the most relevant as per the program's or individual's needs.

A more personalized and preventive approach to various diseases and conditions and general well-being requires developing a broad fasting and lifestyle regimen for individuals to follow either daily or as per specific need. Using the methods and programs herein will help allow for more informed decisions and better outcomes for individuals. Individuals will be more empowered so that they can take a more proactive role in their lifestyle changes contributing to their growth and healing. The platform will help in understanding the different types of aspects of fasting and lifestyle modification available and the optimal ways these can be combined for improved for individual outcomes.

### Body Fluid Testing

The methods and items disclosed herein also allow a more facile process of determining levels of an individual's biomarkers in bodily fluids. Such a process can be performed by an individual in their own home, by using a test strip and analyzing the test strip with a standard personal device such as a smartphone.

Conventionally, urine strips which are used in diagnostics are either analyzed visually or with a urine analyzer machine. Visual analysis of a home test is done by looking at the color of the bio marker pad on the strip and visually comparing it to the printed chart on the packaging of the strip. In pathology labs, the tests are done by taking urine samples and processing them in a Urine Analyzer machine to derive the results for the Urine Scan.

The visual method suffers from the accuracy of the reading being highly prone to errors due to the fact that it uses visual inspection. Individual ability to do color analysis with a high degree of gradation varies from person to person, and is dependent on vision quality. These inspection skills of a user have a high level of variability. The use of a Urine analyzer machine, on the other hand, suffers from lack of convenience, time delays, and cost.

Additionally, all the methods currently in the market globally only analyze the urine biomarkers for its medically approved ranges. There is no algorithmic or academic analysis done on the urine sample or using strip to map it to Ayurvedic parameters of elements, chakras and doshas and translated into wellness state.

Point of Care Testing (POCT), has been gaining attention for its wide applications in public health. Ranging from blood glucose measurement to complex immunological assays, POCT is simple to use even for end users with little technical expertise. This characteristic makes it particularly suitable for disease detection, quality of care, or patient compliance monitoring. Although advancements in semiconductor technology have helped create transformative consumer devices, so far they have had limited impact on the medical world. Medical devices have stringent requirements for safety and effectiveness, and they generally require bulky, power-hungry designs. One of the leading diagnostic tools of POCT analysis is strip-based colorimetric diagnostic assays. When samples such as urine, blood, or other body fluids are deposited on a test strip, signals are obtained in the form of colors. Evaluation of the changes in colors, which is done by simple human perception, is assumed to be accurate. However, the colors may be perceived differently due to varying light conditions, resulting in diagnostic errors which are likely to affect the medical decision-making of users.

Disclosed herein are methods and items to analyze the Home test based Urine Strip and its biomarkers using a smartphone camera and intelligent image processing-based machine learning coupled with algorithmic formulas to derive accurate results.

For the first time, every urine-based biomarker has been mapped with an Ayurvedic elements, doshas, sub-doshas, 5 elements, gunas, dhatus and other variables. Each range of the urine biomarker is also utilized to understand the stress of the body based on the Ayurvedic map.

Once the Ayurvedic doshas/elements/chakras are determined using the algorithm developed under this disclosure, tailored recommendation can be given to the users for balancing their doshas or elements for a holistic living.

This will provide a huge value to the user who can take this test at home and immediately on mobile using a mobile app. Biomarker readings are displayed along with their significance to daily lifestyle for balanced living.

### Disease Detection and Prevention

The methods and items disclosed herein can be used to detect or diagnose diseases in an individual or patient. The information from patient questions and/or measurement of biomarkers can be algorithmically mapped to detect or diagnose diseases, even if the disease is not otherwise evident. In some cases, the algorithmic mapping can be by using Ayurvedic principles. The diseases diagnosed can be, for example, disorders of the kidney, liver, endocrine system (e.g., diabetes), intestine, gall bladder, and cholesterol levels. In some cases, the methods and items disclosed herein can be used to detect likelihood of a future disease occurring, for example, that the patient is predisposed to a certain disease state, or that the patient is showing signs of progressing towards a disease state, e.g., the patient is prediabetic or prehypertensive.

Once a disease or predisposition to a disease is detected or diagnosed, the methods of the disclosure can be used to design a treatment regimen for the patient, and the patient can be treated to prevent, cure, ameliorate, alleviate, or reduce symptoms of the disease.

### Medicinal Treatment

The methods and items disclosed herein can be used to design medicinal treatment, e.g. use of pharmaceutical, nutraceutical, or herbal medicines, to treat the patient.

In some embodiments, the body fluid testing and answers to patient questions can be processed by algorithmic mapping using Ayurvedic principles, as discussed throughout the disclosure, to design a treatment regimen. For example, determination of imbalances of Ayurvedic elements and chakras can be used to design a course of treatment with Ayurvedic herbal medicines to be administered to the patient. Combination of such medicinal treatment with diet and lifestyle regimens, such regimens also determined by the methods herein, will provide beneficial holistic treatment to the patient.

### EXAMPLES

### Example 1. Urine Test Strip

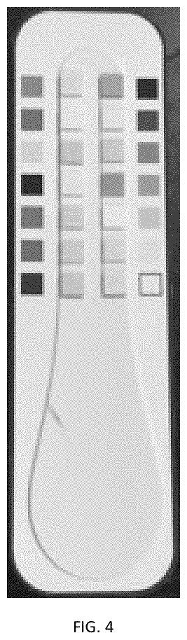

A urine test strip is prepared with up to 14 urine markers. One version of the strip has patches to measure the following biomarkers: urobilinogen, bilirubin, ketone, creatinine, protein, micro albumin, glucose, nitrite, leukocytes, specific gravity, pH, ascorbate, calcium, and blood. One embodiment of the test strip is depicted in FIG. 4,

The test strip is exposed to a patient's urine, and the colors of the exposed patches are imaged using the camera of a smart phone, where the smartphone has an analysis app installed to analyze the patch colors. The patch colors are analyzed and converted into biomarker levels. The biomarker levels are then converted into a derangement status based on the extent to which each biomarker deviates from a predetermined normal level. Sample derangement statuses are normal, stress, high stress, and toxicity. Derangement status for each biomarker can be converted into an imbalance status for Ayurvedic doshas, elements, and/or chakras. Imbalance status can be stated as numbers (e.g., from 1 for the least to 4 for the greatest). Imbalance status can then be used to design a fasting and lifestyle regime using algorithmic mapping.

Derangement statuses/Stress Levels are defined based on the biomarker reading range, and points are allocated for the degree of stress. For example, 1: normal; 2: stress observed; 3: high stress observed; 4: toxicity (detox advised).

For every biomarker, elements responsible for the derangement of the readings are already mapped in their priority of “Imbalance causing” of that marker from high to low.

For every biomarker, points are given to all five elements based on the degrees of Stress Levels found from the quantitative measurement of the urine strip

For all biomarkers, gross totals should be calculated for every element (earth, water, fire, air and ether). The maximum scored element/chakra should be treated first for the imbalance followed by the second highest scored element, and so on.

### Example 2. Fasting Defined

What Types?

Strengthening Body:
Intermittent-Meal, Liquid-Meal, Fruits-Munch
Detox-friendly, Nutrition-Rich, Medicinal

Streamlining Mind:
Facing Hunger, Pacifying Hunger, Feasting on Meditations
Fear-Detox, Anger-Detox, Sex-Detox

Soaring Soul:
Special-Meal, Just-Water, Body-Sensing
Loving Sun, Creative Marathon, At-Work-24×1

### Fasting Types: Briefly Defined

Strengthening Body: One-Day Programs
Intermittent-Meal: No food for 12-18 hrs. Core Feature(s): Set time, duration and notifications. (e.g. 8 pm to 8 am)
Liquid-Meal: Only Liquid/Semi-Liquid foods. C F: Diet suggestion and recipes.
Fruits-Munch: Only fruits (liquid, cut, dried or may be cooked). C F: Diet suggestion and recipes.
Detox-friendly: All foods that helps detox one or all parts. C F: Diet suggestion based on Detox-type selection or last profile update.
Nutrition-Rich: All foods rich in variety of nutrition. C F: Diet suggestion based on latest profile and diagnostics (if taken and entered).
Medicinal: Specific foods and medicinal herbs only. C F: Diet suggestion based on latest profile and diagnostics (if taken and entered).

Streamlining Mind: One-Day Programs
Facing Hunger: Consuming foods & content to identify different types of hungers. C F: Suggestion of specific diet/fasting program, breathing, audio-visual.
Pacifying Hunger: Special content to pacify specific hunger and control it. C F: Suggestion of special fasting program, breathing and audio-visual.
Feasting on Meditations: Special content with balancing diet. C F: Suggestion of special diet, breathing and audio meditations.
Fear-Detox: Special content with balancing foods. C F: Suggestion of special diet, breathing and audio-visual meditations/content.
Anger-Detox: Special content with balancing foods. C F: Suggestion of special diet, breathing and audio-visual meditations/content.
Sex-Detox: Special content with balancing foods. C F: Suggestion of special diet, breathing and audio-visual meditations/content. Soaring Soul: one-week programs
Special-Meal: Satvic food only. C F: Suggestion of diet program and meditations.
Just-Water: Only water, no food (intermittent). C F: Set time and duration. Suggestion of meditation and breathing program.
Body-Sensing: Satvic food and special content to help sense different parts of the body and ego. C F: Special programs with content, diet, yoga and meditation.
Creative Marathon: Balancing foods and special exercise to increase focus and creativity. C F: Set time, duration, notifications. Suggestion of meditation program.
At-Work-24×1: Balancing foods and special exercise to increase focus and strength. C F: Suggestion of special breathing, meditation and diet program.
Loving Sun: Gazing at the rising Sun with Satvic food, energized water. C F: Set time and notification; automated timer based on time+UV.

Suggestion of special daily practice/program (Sun, food, water, content).

### Example 3. Primary Pathway

Examples of questions to be asked of the patient:

Who are you? (basic identifiers)

How can we help you? (user observation of self/life)

We believe, you are: (Ayurvedic profile)

This Fasting program may suit you.

Would you like to customize this program or select one yourself?

Your Fasting History//Your Profile+Feedback

### Example 4. Meditations

Below are sets of meditations for use in some embodiments of the methods disclosed herein

Set 1: Escape from Tamas

Set 2: Rising into or out-of Rajas

Set 3: Reaching or enriching Sattva

(multiple variations of movements and mix)
set 1+2+3=total variations TBD

Guided Meditations:

The following provides direction, based on identifiable categories. This simply points to the starting point.

Further expansion should take place before or soon after launch of the service.

Set 4: Detoxification (as many variations of the categories below)

Anger:
liquefying anger
expressing anger
pacifying anger
observing the rise of the anger within
observing the anger entering the aura, into self
observing the anger jumping out without notice
facing/controlling anger
surrendering the anger to the Divine
flooding Anahata with higher love

Fear:
discovering fear(s) in the subconscious
observing fear's rising and churning into mind; body
facing the fear and clearing it from the roots
replacing fear with courage
strengthening Muladhara
strengthening Swadhishthana
strengthening Manipura
taking refuge in open Anahata
surrendering fear to the Divine

Sex:
calming of Swadhishthana (dull underlying overtones of desire vanishing into crisp peace)
balancing Vishuddha (evolution of speech from slight stutter to clear expression and crisp sound)
energizing Anahata (heart being brought into its innate nature of wideness and embrace)
battle no more (from sounds of battle to control to winning the truest freedom)
glimpse of the true love
experiencing the true oneness
surrendering the desire to higher self
recovering the memory of sacred union
opening to the Divine Master
opening to the Divine Mother
falling in love with Divine
the one and only: the Divine

Set 5: Pacifying Hunger:
calming body
calming vital; clearing vital
emptying mind
recovery and balance
discovering soul
quenching the soul
gazing at the soul
staying in the bliss
experience of being fed by the Divine love
ultimate transcendence
visualization of new body of light
   set 4+5=total 41 (+variations)

Lectures/Dynamic Meditations

Energizing and Enriching Mind

Set 6: Facing Hungers:
defining desire, defining hunger
understanding manifestation and existence of hunger
basic need becoming desire
physical, vital & mental play in hunger(s)
arresting the impulse in its play and value
will of the mind vs. will of the spirit
spiritualization of the hunger
hunger and the sacred fire
the role of present human body
evolving into new body of light

Set 7: Creative Marathon:
creative vs. pro-creative impulse
discovering innate expression
strengthening the vital
removing blockages and improving the flow
flowering the Vishuddha chakra and Swadhisthana
union of the two chakras
ways of visualization
spurring the process of visualization
blooming of clear vision
experiencing the Divine ecstasy

Set 8: Body-Sensing:
going within and locating awareness
strengthening the nerves
focusing and deepening connection in <the part/organ/limb> (each and all)
descent from above through the spine, into the earth
ascent from Muladhar and discovering refuge in <the chakra> (each of top 4)
journeying and flowing freely throughout the body
flooding the body with the Divine light

Set 9: At Work, In Love:
Discovering vigor within
Sharpening focus
Deepening the focus
Ways of escaping the brain-fog
Connecting with the body
Staying in rapture without fatigue
   set 6+7+8+9=total 33 (+variations

### Example 5. Music and Stories

Set 1 (b): Escape from Tamas (m)

about 7 min (in a pair of elements, the first being primary, the elements-mix-ratio may be about 5:2)

Set 2 (m): Rising into or out-of Rajas (m)

about 7 min (in a pair of elements, the first being primary, the elements-mix-ratio may be about 5:2)

Set 3 (s): Reaching or enriching Sattva (m)

minimum 10 min (multiple variations of movements and mix)
set 1+2+3=total 30 (+variations)

The following provides direction for music as well as stories, based on identifiable categories.

This simply points

to the starting point. Further expansion should take place before or soon after launch of the service.

Set 4: Detoxification (as many variations of the categories below)

Anger: (m+s)
liquefying anger
expressing anger
pacifying anger
observing the rise of the anger within
observing the anger entering the aura, into self
observing the anger jumping out without notice
facing/controlling anger
surrendering the anger to the Divine
flooding Anahata with higher love

Fear: (m+s)
discovering fear(s) in the subconscious
observing fear's rising and churning into mind; body
replacing fear with courage
strengthening Muladhara
strengthening Swadhishthana
strengthening Manipura
taking refuge in open Anahata
surrendering fear to the Divine

Sex: (m+s)
calming of Swadhishthana (dull underlying overtones of desire vanishing into crisp peace)
balancing Vishuddha (evolution of speech from slight stutter to clear expression and crisp sound)
energizing Anahata (heart being brought into its innate nature of wideness and embrace)
battle no more (from sounds of battle to control to winning the truest freedom)
discovering true love
experiencing the true oneness
surrendering the desire to higher self
recovering the memory of sacred union
opening to the Divine Master
opening to the Divine Mother
falling in love with Divine
the one and only: the Divine

Set 5: Pacifying Hunger:
calming body
calming vital; clearing vital
emptying mind
recovery and balance
discovering soul
quenching the soul
gazing at the soul
staying in the bliss
experience of being fed by the Divine love
ultimate transcendence
visualization of new body of light
   set 4+5=total 41 (+variations)

Music for Lectures/Stories (Dynamic Meditations):

Energizing and Enriching Mind

Set 6: Facing Hungers:
defining desire, defining hunger
understanding manifestation and existence of hunger
basic need becoming desire
physical, vital & mental play in hunger(s)
arresting the impulse in its play and value
will of the mind vs. will of the spirit
spiritualization of the hunger
hunger and the sacred fire
the role of present human body
evolving into new body of light

Set 7: Creative Marathon:
creative vs. pro-creative impulse
discovering innate expression
strengthening the vital
removing blockages and improving the flow
flowering the Vishuddha chakra and Swadhisthana
union of the two chakras
ways of visualization
spurring the process of visualization
blooming of clear vision
experiencing the Divine ecstasy

Set 8: Body-Sensing:
going within and locating awareness
strengthening the nerves
focusing and deepening connection in <the part/organ/limb> (each and all)
descent from above through the spine, into the earth
ascent from Muladhar and discovering refuge in <the chakra> (each of top 4)
journeying and flowing freely throughout the body
flooding the body with the Divine light

Set 9: At Work, In Love:
Discovering vigor within
Sharpening focus
Deepening the focus
Ways of escaping the brain-fog
Connecting with the body
Staying in rapture without fatigue
   set 6+7+8+9=total 33 (+variations)

### Example 6. App Design

### Major Features

1. Individual Profiling based on Earth Elements for Fasting Recommendations

2. Ayurvedic Science (Natural healing techniques) Mapped Algorithm for Recommendations of Fasting Program

3. Programmatic Design and Integration for Wellness Impact based on Ayurveda by Solar and Lunar Cycles (geo/time) for food, meditation, breathing, sound/audio, stories

4. All in to APP For Mobile platform

### STEP 1. Individual Profiling based on Earth Elements for Fasting Recommendations

Based on series of questions and answers (multiple choices) a highly intelligent algorithmic formulation is provided to create a profile of an individual.

The questions capture physiological, biological, mental and spiritual states using Q&A.

It looks deeply analyzes imbalances and abnormalities in day to day living of an individual based on Q&A feedback

A deep Database is mined to develop patterns and grids to get accurate snapshot of the following:

a. Element Mapping of individual (earth, water, air, ether, fire)

b. Based on Elemental mapping it creates a snap shot of natural state (Three DOS HAs—Vata, Pitta, Kapha)

c. Based on the combination of both, a current state profile is create for the individual

### STEP 2. Ayurvedic Science (Natural Healing Techniques)

### Mapped Algorithm for Recommendations of Fasting Program

Science is developed to understand Human Nature, State and Characteristics (physical, mental, biological, spiritual). Our algorithms have mapped this into machine learning science. Based on the profile, the geography, the time of the month: a sophisticated mapping of specific fast program is created for the individual. within each fast, specific DIET, MEDITATION, BREATHING, SOUNDs are suggested to map to the vibrational frequency of each individual state as mapped from the profile. A guided program is generated for a day, week, month, based on the need of the individual. The program measures, maps and modulate by looking deeply and analyzing imbalances and abnormalities in day to day living of an individual.

Programmatic Design and Integration for Wellness Impact based on Ayurveda by Solar and Lunar Cycles (geo/time) for food, meditation, breathing, sound/audio, stories

### Example 7. App Flow

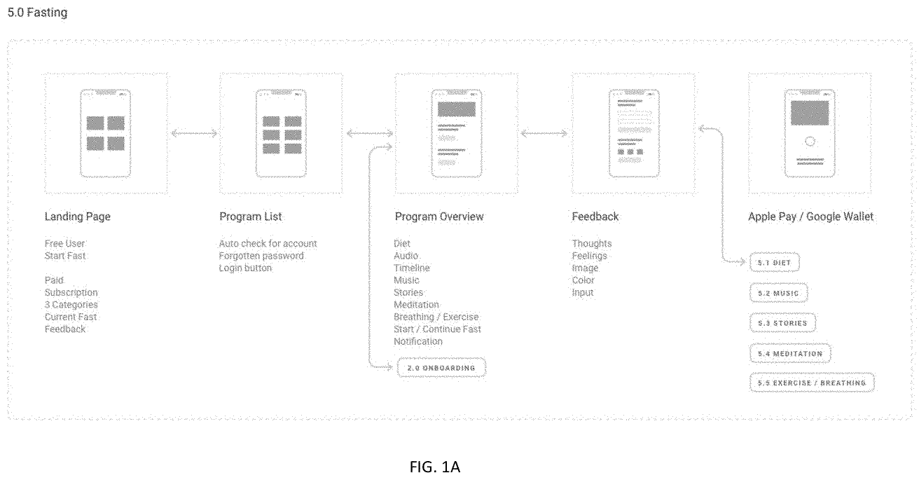

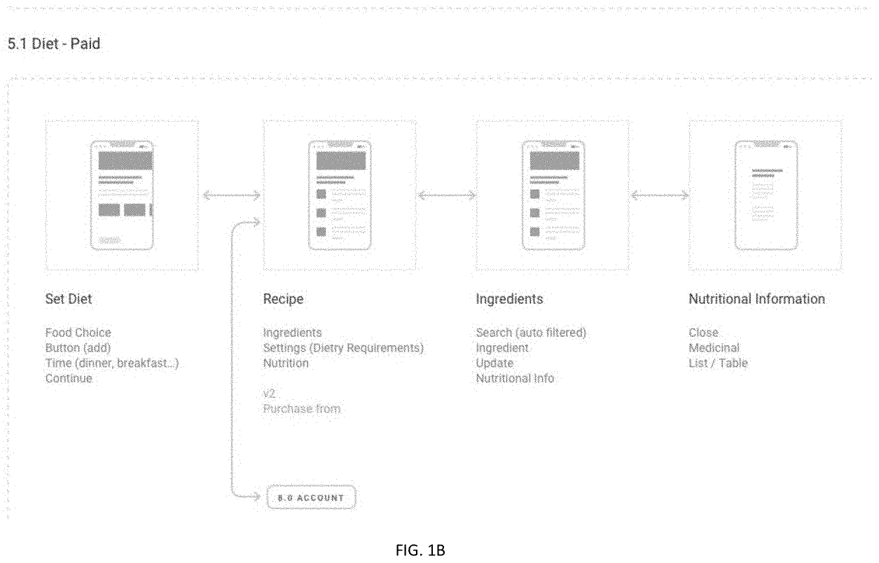

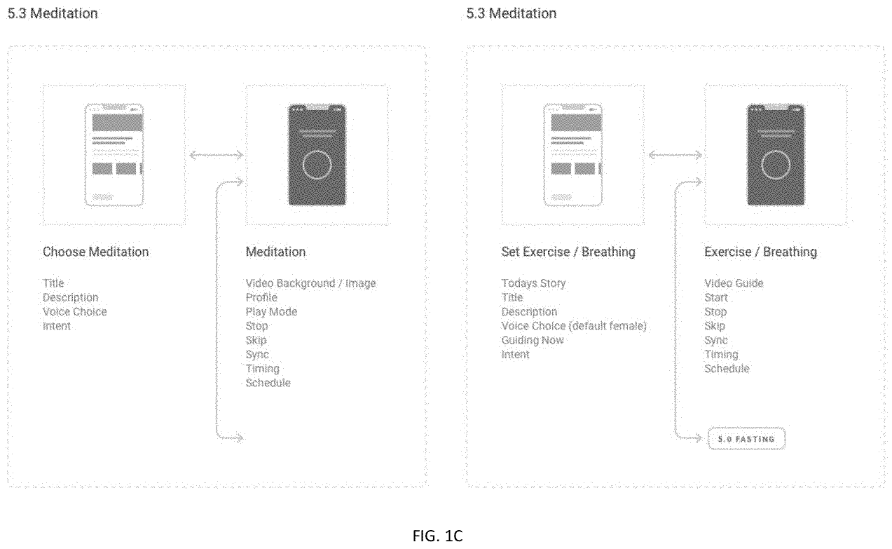

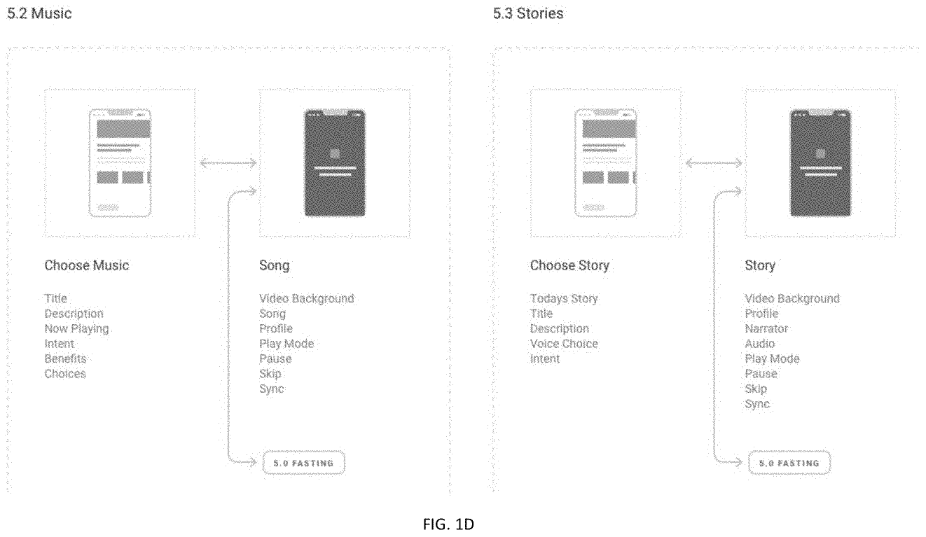

The app flow is shown in FIGS. 1A, 1B, 1C, and 1D. FIG. 1A depicts the overall app structure and app flow for a mobile app for designing and monitoring a fasting and lifestyle regimen. FIG. 1B depicts the app flow for the diet element of the app. FIG. 1C depicts the app flow for the meditation element of the app. FIG. 1D depicts the app flow for the music and story elements of the app.

### Example 8. App Features and Functions

This example illustrates parts of the menus, features, and functions of an embodiment of the mobile app for designing and monitoring a fasting and lifestyle regimen

### Visitors:

### A New

B. Enrolled & Passive (weekly/monthly visits)
C. Enrolled & Active (daily/frequent visits)

### Bottom Bar/Global Menu

Fasting: guided fast VS. self-select (PHASE 2 feature: fasting-glossary)
Now: current program/one just finished/next fast-type suggestion or scheduled
History: total fasts+types evolution of strength (PHASE 2 feature: wellness/balance graph)
Profile: BioPhysical Profile (with elements+glossary); Feedback; Settings, Account ID
Culture (PHASE 2 features: wisdom, wellness community)

### App Screens and Basic Behavior

### Launch Screen

### Splash Screen

### Home

Identifying the Visitor>milestone (if, active)
Presenting one of the 3 screens:
Fasting: Categories & Types/Now: Current Program/Profile:Your Elements &
Nature (date)
(profile/test presented to the AleW users, or to the ‘Enrolled’ to promote monthly/weekly update.)

### Fasting

2 paths: Guided Fast VS. Self-Select
If not an advanced user, Guided Fast presented. For advanced user, both options.
Guided Fast:
Landing page>Categories & Fasting Types/Programs:
   Strengthening Body (5 types)
   Streamlining Mind (6 types)
   Souring Soul (5 types)
   If an advanced user, suggestion for Self-Select.
   If not an advanced user, one category>program from the list highlighted.

### Self-Select

Landing page>Categories & Fasting Types/Programs
   Step 1-5:
   Selection of fast-program>Selection of Music, Stories, Mediations>Naming the Program>
   Setting time, duration, event-repeat+notifications>Q. take test/profile before start<yes/no>
   (custom programing for alarms/timer except for the program: Loving Sun)
   If not an advanced user, warning message. <Skip/Guide Me>

### Now

Identify the milestone of the user's program/practice
Presenting one of the 3 screens:
current program/the one finished/next fast-type/program suggestion

### History

Landing Page>Summary
Detail View:
   your commitment: # Fasting Programs, # Finished/Date, Time
   your constitution: changes noted based on Ayurvedic profile
      If not submitted new profile before fasting program, message presented to express
      the system's inability to register. Instead, the last registered profile presented with <Update>.

### Profile

Landing Page>BioPhysical Profile+<Update>/Feedback (only if they just finished a program)
BioPhysical Profile (multiple copies stored by date)
   Bio-Data (Height, weight etc.)
   Elements & Nature (Ayurvedic profile)
Feedback (presented upon finishing fasting)
   Your thoughts (Q/A: words/phrases to be selected)
   Your feelings (Q/A: images to be selected)

### My Settings

### Profile:

(a) registration and on boarding of the new user
(b) once profiled, editing or updating profile information
(c) once participated, taking in feedback
(d) my settings
   Registration+On-boarding the user for Bio-Scan
Create
Registration (ID: name, email/mobile, password)
Bio-Data
Multi-choice step by step Q&A to create profile of the user based on 5 fundamental elements
(This would log the basic nature/condition of the user+date of submission−storing multi-copies for history)
Save
Edit
Summary Card of this profile
Link to description of 5 elements (scientific & poetic)+glossary

### Create and Update

Account ID
Mobile # or Email ID
Set Password
Verify Password via email
Update Password
Bio-Data
Age
Gender
Height
Weight
User Level
(Explorer/Disciplined/Advanced Seeker—noted by the system per participation and completion of programs)

### Settings

Enable Notification
Allow Location Use

### Feedback

Q&A for thoughts: store answers and verify profile assumptions
Q&A for feelings: store answers and verify profile assumptions

### Fasting:

Main Categories>Types:
Strengthening Body
Two Paths:
Guided VS. Self-Select
   Listing Programs for each category
   Program Review: Understand a fasting program (audio)
      Allows user to understand what is the fast about, benefits, total fasting time, and the step by step process to undertake breathing/exercise, meditation, music, stories, diet etc.
   Select/Design a fasting program
      Activate a “Single” fasting program
         Today/Now
         Later by date/month and time
         On-time or Recurring event selection
      Summary Screen of Program

### Now.

Where they are in the program (step 4 of 6)
What is next and onwards during the day (step 5, 6)
   Notifications
      Okay and continue
      Postpone to ‘Remind in <x> minutes’
      Cancel to stop the program

### Fasting>Categories>Types/Programs:

Strengthening Body. one-day programs
Intermittent-Meal:
No food for 12-18 hrs. Core Feature(s): Set time, duration and notifications. (e.g. 8 pm to 8 am)
Liquid-Meal:
Only Liquid/Semi-Liquid foods. C F: Diet suggestion and recipes.
Fruits-Munch:
Only fruits (liquid, cut, dried or may be cooked). C F: Diet suggestion and recipes.
Detox-friendly:
All foods that helps detox one or all parts. C F: Diet suggestion based on Detox-type selection or last profile update.
Nutrition-Rich:
All foods rich in variety of nutrition. C F: Diet suggestion based on latest profile and diagnostics (if taken and entered).
Medicinal:
Specific foods and medicinal herbs only. C F: Diet suggestion based on latest profile and diagnostics (if taken and entered).

### Fasting>Program Execution

Fasting program is an automated task which will use notification engine to initiate each step in the program.
User will stay engaged by selecting “okay” or “remind/later” and accomplish the each program-step accordingly.
On each step, the program may offer one or a combination of the following recommendation(s):
Music
Recommendation List (tailored by fast type and unique bio-physical profile+user preference)
Breathing Exercise
Recommendation List (tailored by fast type and unique bio-physical profile)
Food
Recommendation List (tailored by fast type and unique bio-physical profile)
Meditation
Recommendation List (tailored by fast type and unique bio-physical profile+user preference)
Stories
Recommendation List (tailored by fast type and unique bio-physical profile+user preference)

### Program>Content

Consumption recommendations tailored by fast type and unique bio-physical profile.
Music
   List of recommendations
   Summary card of each item
   intent and benefits
   Select (from recommendations list) or Accept (suggestion if Guided Fast)
   Play and Pause
   Display moving image on screen
   Skip
Breathing/Exercise
   Advanced or not, the user cannot modify the suggestion.
   Summary card for each exercise
   intent and benefits
   Animated graphic for special breathing cycle and pattern
   Message+Sound/Vibrate at Start and end
   Accept (okay)
   Play and Pause
   Skip
Stories
   Summary card for each story
   intent and benefits
   Select (from recommendations list) or Accept (suggestion if Guided Fast)
   Play and Pause
   Display moving image on screen
   Skip
Meditation
   Summary card for each story
   intent and benefits
   Select (from recommendations list) or Accept (suggestion if Guided Fast)
   Play and Pause
   Display moving image on screen (if open-eye meditation)
   Skip
Food
Advanced or not, the user can select/modify the diet suggestion.
   Select (from recommendations list)
   Recommendation Categories
      By Meal time (lunch, dinner, snacks etc.)
      By Meal type (sandwich, soup etc.)
      By Preference (Vegan, Liquid, Medicinal etc.)
   Selected Meal>
      Display Meal options by Recipe
      Show Constitutional Benefits (per science/insight of Ayurveda)
      Ingredient List (option to choose up to three alternative ingredients)
      Multiple recipes to choose from (upto six)
      Display Meal options by Meal Type+ingredient list
      e.g. sandwich with spinach, tomato, avocado
      Display Meal Options by Meal Times
      Breakfast, Lunch, Snacks, Dinner etc.
      For all of the above:
      Ingredient level: Show/display on demand
      Constitutional benefits
      Nutritional facts
      Calories per serving
      Element Type
      Images for Recipes
      Summary card for ingredient

### Notification

### Notifications (Text+Sound/Vibrate)

Notification for:
   Reminder of “Fast” 1 day prior
Ability to:
   Accept
   Remind in <x> minutes
   Skip for later
   Cancel

### Notifications:

will be on Lock screen and within application
   Each Fast Program to be designed and developed by internal doctors (in progress)
   Content Database (strategy, design and development in progress)

### Ayurveda Database

Ready to use with all ingredients, nutrition and tags (5 elements, doshas, effects etc.)
along with
Meal type, Meal time and other ideal consumption conditions
Medicinal properties database ready
Recipes database in progress

### Example 9. Dietary Summary Cards

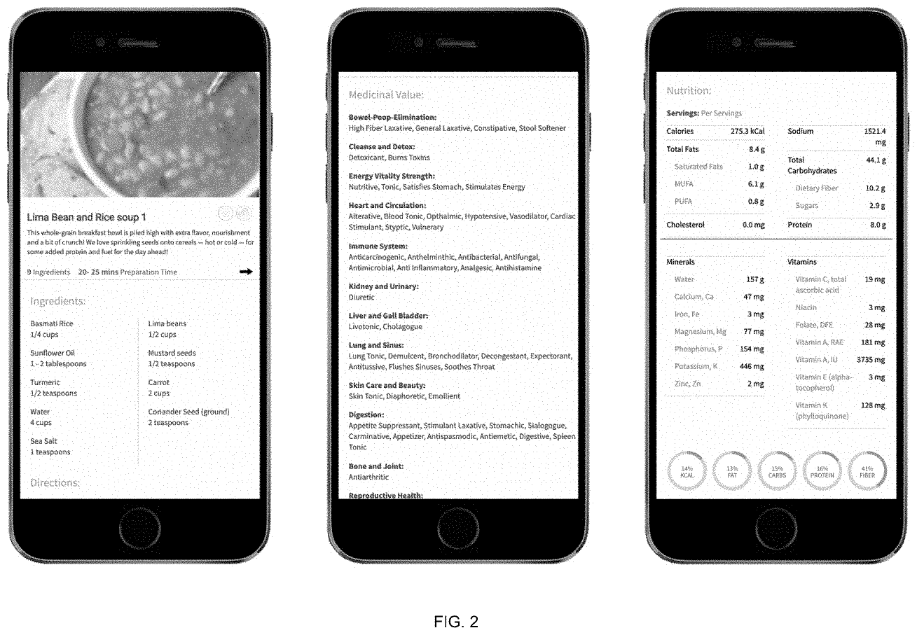

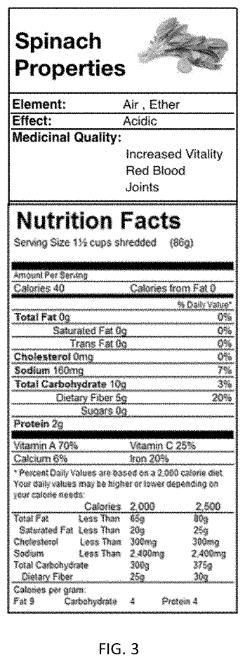

The app may contain information on dietary components such as ful recipes or individual ingredients. FIG. 2 depicts a sample summary card for a recipe (lima bean and rice soup). FIG. 3 depicts a sample summary card for an ingredient (spinach).

### Example 10. Ayurvedic Charts

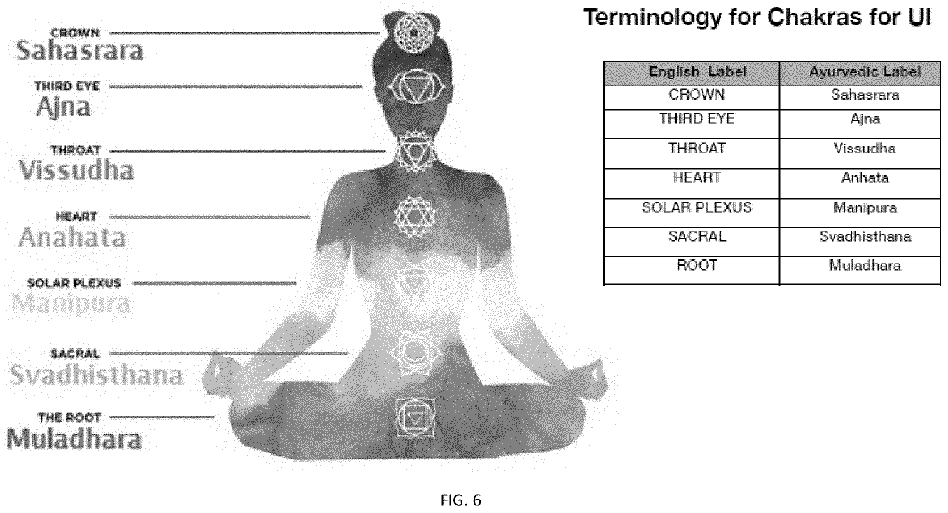

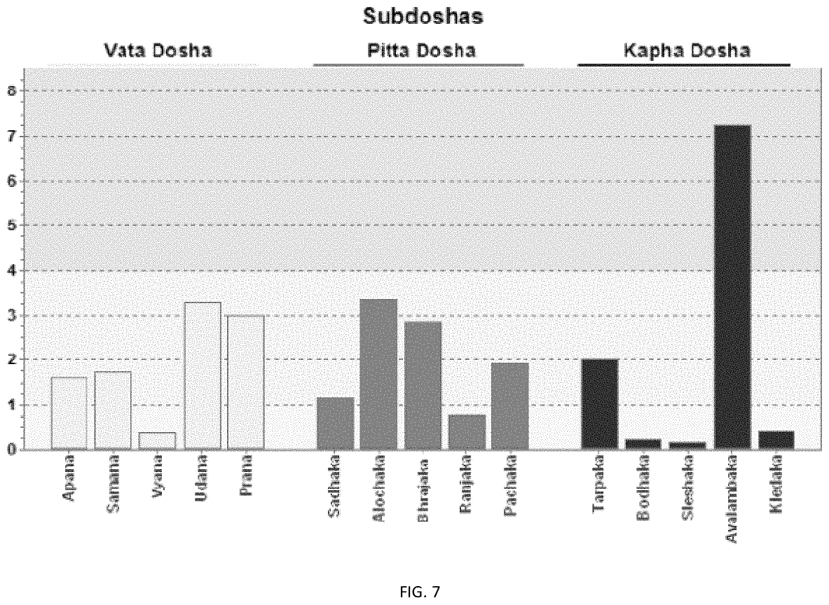

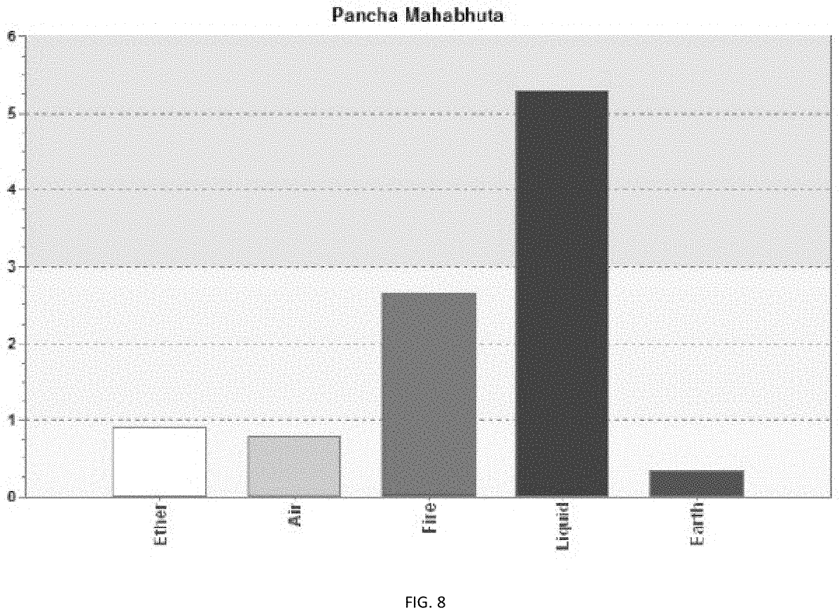

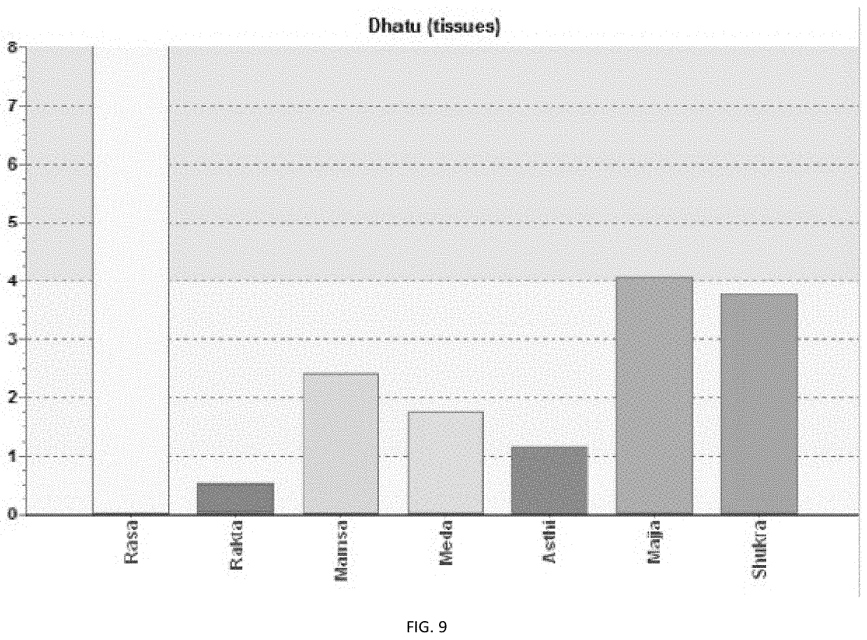

A few sample ayurvedic charts are provided to show relationships of Chakras, Doshas, tissues, and elements. FIG. 6 depicts labels and positions of Chakras. FIG. 7 depicts levels of subdoshas in each Dosha. FIG. 8 depicts an example of the degree to which individual ayurvedic elements are contributing to a deranged biomarker in a subject. FIG. 9 depicts an example of the degree to which individual tissues are in imbalance in a subject.

### Further Embodiments and Principles

The “plafform”, as used herein, can refer to an app (i.e., an application), computer program, etc. The plafform can be used on a mobile device, computer, or other device containing a processor.

In some implementations, the systems and techniques described here can be implemented in a computing system that includes a single computing device in communication with other computing devices, or that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (“LAN”), a wide area network (“WAN”), and the internet.

The computing system can include clients and servers. A client and server are generally remote from each other and can interact through a communication network. The relationship of client and server arises by virtue of computer programs running on the respective computers and having a client-server relationship to each other. In some embodiments, a server transmits data (e.g., image data) to a client device (e.g., for purposes of displaying data to and receiving a response to an operation performed on the data). Data generated at the client device (e.g., a result of the user interaction) can be received from the client device at the server.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

Embodiments of the subject matter and the operations described in this specification can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer programs, i.e., one or more modules of computer program instructions, encoded on computer storage medium for execution by, or to control the operation of, data processing apparatus.

Alternatively or in addition, the program instructions can be encoded on an artificially generated propagated signal, e.g., a machine-generated electrical, optical, or electromagnetic signal, that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media (e.g., multiple CDs, disks, or other storage devices).

The operations described in this specification can be implemented as operations performed by a data processing apparatus on data stored on one or more computer-readable storage devices or received from other sources. The term “data processing apparatus” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a standalone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language resource), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

While this specification contains many specific implementation details, these should not be construed as limitations on the scope of any inventions or of what may be claimed, but rather as descriptions of features specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination. Thus, particular embodiments of the subject matter have been described. Other embodiments are within the scope of the following claims. In some cases, the actions recited in the claims can be performed in a different order and still achieve desirable results.

1. A method for designing and monitoring a fasting and lifestyle regimen for an individual, the method comprising:
a) Diagnosing the individual by a series of questions and physical measurements to determine a health-constitution (test) profile;
b) Determining one or more goals to achieve balance by analyzing the test profile of the individual or by accepting direct input of the individual;
c) Determining a fasting and lifestyle regimen for the individual by algorithmic mapping of the test profile and one or more goals directed to achieve balance and growth;
wherein the fasting and lifestyle regimen comprises specifying
(i) a duration and method of use of complete program; and
(ii) type and amount of food to be eaten when the individual is not in complete fasting.

2. The method of claim 1, the method further comprising:
d) Determining timing of each activity based on Ayurvedic time scale and solar/lunar cycles;
e) Determining element-based mapping of all activity during fasting and for daily life to help maintain wellness; and
f) Mapping integrated database to power the fasting and lifestyle regimen using Dosha, Elements, Chakras, Seasons, Characteristics, Smell, Taste, Volume and Method of Use.

3. The method of claim 1, wherein the fasting and lifestyle regimen comprises specifying
(i) a duration and method of use of complete program;
(ii) type and amount of Ayurvedically mapped food to be eaten when the individual is not in complete fasting; and
(iii) one or more modules selected from the group consisting of: music therapy using ayurvedic principles, pranayama breathing exercises, customized integral yoga meditation per a program goal, contemplative story narrations, story/talks listening, and visual video therapy and movement routines to be performed by the individual.

4. The method of claim 1, comprising:
a) Diagnosing the individual by a series of questions and physical measurements to determine a health-constitution (test) profile;
b) Determining one or more goals to achieve balance by analyzing the test profile of the individual or by accepting direct input of the individual;
c) Determining a fasting and lifestyle regimen for the individual by algorithmic mapping of the test profile and one or more goals directed to achieve balance and growth;
d) Determining timing of each activity based on Ayurvedic time scale and solar/lunar cycles;
e) Determining element-based mapping of all activity during fasting and for daily life to help maintain wellness; and
f) Mapping integrated database to power the fasting and lifestyle regimen using Dosha, Elements, Chakras, Seasons, Characteristics, Smell, Taste, Volume and Method of Use;
wherein the fasting and lifestyle regimen comprises specifying a duration and method of use of complete program;
(ii) type and amount of Ayurvedically mapped food to be eaten when the individual is not in complete fasting; and
(iii) one or more modules selected from the group consisting of: music therapy using ayurvedic principles, pranayama breathing exercises, customized integral yoga meditation per a program goal, contemplative story narrations, story/talks listening, and visual video therapy and movement routines to be performed by the individual.

5. The method of claim 1, wherein the algorithmic mapping is performed using Ayurvedic principles and sciences, wherein mapping comprises emotional mapping, physical mapping and mental mapping, and wherein physical measurements comprise chemistry and composition of urine, saliva and other body fluids of the individual.

6. The method of claim 1, wherein the fasting and lifestyle regimen comprises specifying each of music listening, breathing exercises, meditation, story reading, and story/talk listening.

7. A system for designing and monitoring a fasting and lifestyle regimen for an individual, the system comprising a processor and a memory storing instructions that, when executed by the processor, provide an application that performs the following steps:
a) Collecting a first set of user responses to a series of questions and physical measurements to determine a test profile;
b) Determining one or more goals to achieve balance by analyzing the test profile of the individual or by accepting direct input of the individual;
c) Determining a fasting and lifestyle regimen for the individual, determination comprising algorithmic mapping of the test profile and one or more goals directed to achieve balance;
d) Determining timing of each activity based on Ayurvedic time scale and solar/lunar cycles, and an Ayurvedic element map;
e) Determining element-based and chakra-based mapping of all activity during fasting and for daily life to help maintain wellness;
f) Displaying the actions suggested by the regimen;
g) Tracking and displaying the individual's progress in following the regimen; and
h) Accessing a fully mapped integrated database to power the fasting and lifestyle regimen using Elements, Chakras, Doshas, Seasons, Characteristics, Smell, Taste, Volume and Method of Use;
wherein the fasting and lifestyle regimen comprises specifying a duration of complete fasting;
(ii) type and amount of Ayurvedically mapped food to be eaten when the individual is not in complete fasting; and
(iii) one or more modules selected from the group consisting of: music listening/therapy, breathing exercises, custom meditation per the program goal, story reading, story/talks listening, and visual video therapy and movement routines to be performed by the individual.

8. The system of claim 7, wherein tracking and displaying the individual's progress comprises collecting one or more second sets of user responses to a series of questions and physical measurements, to determine a progress profile; and using said progress profile to create a modified fasting and lifestyle regimen.

9. The system of claim 7, further comprising algorithmic mapping for deep therapeutic application at a Mind, Body and Soul level to deal with emotions (e.g., anger, fear, etc.), creativity (e.g., intuition, insight, etc.), desires (e.g., sex, jealousy, hate, etc.), physical development (e.g., body vitality, etc.), consciousness (e.g., introspection, contemplation, etc.) and other yogic practices such as SUN Gazing, and Water Fasting.

10. The system of claim 7, wherein collecting a first set of user responses to a series of questions and physical measurements comprises collecting a body fluid sample from the individual, and wherein analyzing the body fluid sample produces a body fluid test profile.

11. The system of claim 10, wherein analyzing the body fluid sample comprises colorimetric analysis of one or more body fluid test strips to determine levels of one or more biomarkers and thereby produce a body fluid test profile.

12. The system of claim 11, wherein colorimetric analysis of the one or more body fluid test strips is performed using a mobile device.

13. The system of claim 12, wherein the application executes on a device comprising a camera, and colorimetric analysis of the one or more body fluid test strips comprises
a) taking an image of each test strip using the camera;
b) analysis of the image by the mobile app to determine color hue and/or color density of portions of the test strip, and
c) correlation of the color hue and/or color density with levels of one or more biomarkers to determine levels of each biomarker and thereby produce a body fluid test profile.

14. The system of claim 10, wherein the body fluid is urine or blood.

15. The system of claim 14, wherein the body fluid is urine.

16. The system of claim 13, wherein algorithmic mapping of the body fluid test profile is used to determine a fasting and lifestyle regimen.

17. The system of claim 16, wherein algorithmic mapping of the body fluid test profile comprises determining for each of one or more biomarkers a derangement status based on the extent to which each biomarker level deviates from a pre-determined normal level.

18. The system of claim 17, further comprising wherein each derangement status is mapped to correlate with an imbalance status of one or more Ayurvedic elements selected from the group consisting of air, earth, fire, water, and ether.

19. The system of claim 17, further comprising wherein each derangement status is mapped to correlate with an imbalance status of one or more Ayurvedic chakras consisting of solar plexus, sacral, root, heart, and throat/third eye/crown.

20. The system of claim 18, wherein one or more imbalance statuses are used to determine a fasting and lifestyle regimen.

21. A urine test strip, wherein the test strip comprises test portions to determine levels of biomarkers suitable for determining a fasting and lifestyle regimen.

22. The urine test strip of claim 21, wherein the biomarkers comprise one or more selected from the group consisting of urobilinogen, bilirubin, ketone, creatinine, protein, micro albumin, glucose, nitrite, leukocytes, specific gravity, pH, ascorbate, calcium, and blood.

23. The urine test strip of claim 22, wherein the biomarkers comprise urobilinogen, bilirubin, ketone, creatinine, protein, micro albumin, glucose, nitrite, leukocytes, specific gravity, pH, ascorbate, calcium, and blood.

24. The urine test strip of claim 21, wherein the test strip is suitable for colorimetric analysis by a mobile application, wherein the mobile application executes on a device comprising a camera, and colorimetric analysis of the test strip comprises
a) taking an image of the test strip using the camera;
b) analysis of the image by the mobile app to determine color hue and/or color density of portions of the test strip, and
c) correlation of the color hue and/or color density with levels of one or more biomarkers to determine levels of each biomarker and thereby produce a body fluid test profile.

25. A method of analyzing body fluid, the method comprising:
a) obtaining a sample of body fluid from an individual;
b) exposing a body fluid test strip to the body fluid;
c) taking an image of the test strip using a camera which is part of a mobile device;
d) analysis of the image by a mobile application execution on the mobile device to determine color hue and/or color density of portions of the test strip, and
e) correlation of the color hue and/or color density with levels of one or more biomarkers to determine levels of each biomarker and thereby produce a body fluid test profile.

26. The method of claim 25, wherein the body fluid is urine.

27. The method of claim 25, wherein algorithmic mapping of the body fluid test profile is used to detect a disease state present in the individual.

28. The method of claim 25 wherein algorithmic mapping of the body fluid test profile is used to determine a treatment to prevent development of a disease in the individual.

29. The method of claim 25, wherein algorithmic mapping of the body fluid test profile is used to determine a fasting and lifestyle regimen for the individual.

30. The method of claim 25, wherein algorithmic mapping of the body fluid test profile is used to determine a medicinal treatment for the individual.

31. The method of claim 30, wherein the medicinal treatment comprises administering Ayurvedic herbal medicine.

32. The method of claim 25, wherein algorithmic mapping of the body fluid test profile comprises determining for each of one or more biomarkers a derangement status based on the extent to which each biomarker level deviates from a pre-determined normal level.

33. The method of claim 32, further comprising wherein each derangement status is mapped to correlate with an imbalance status of one or more Ayurvedic elements selected from the group consisting of air, earth, fire, water, and ether.

34. The method of claim 32, further comprising wherein each derangement status is mapped to correlate with an imbalance status of one or more Ayurvedic chakras consisting of solar plexus, sacral, root, heart, and throat/third eye/crown.
